(19) United States
(12) Patent Application Publication
(10) Pub. No.: US 2025/0017518 A1
(43) Pub. Date: Jan. 16, 2025

(54) METHODS AND SYSTEMS FOR PHYSIOLOGICAL DETECTION AND ALERTING

(71) Applicant: THE JOHNS HOPKINS UNIVERSITY, Baltimore, MD (US)

(72) Inventors: Samyak Mehul SHAH, Baltimore, MD (US); Nathan E. CRONE, Silver Spring, MD (US); Gregory L. KRAUSS, Baltimore, MD (US)

(21) Appl. No.: 18/713,803

(22) PCT Filed: Nov. 30, 2022
(86) PCT No.: PCT/US2022/051365

§ 371 Date: May 28, 2024

Related U.S. Application Data
(60) Provisional application No. 63/284,891, filed on Dec. 1, 2021.

(51) Int. Cl. A61B 5/00

(52) U.S. Cl. CPC A61B 5/4094; A61B 5/6803; A61B 5/7257; A61B 5/7267; A61B 5/7282; A61B 5/742; A61B 5/746; A61B 2560/0462; A61B 2562/0219

(57) ABSTRACT

A method for physiological detection and alerting is disclosed that includes obtaining biometric sensor data from a user; generating processed biometric sensor data from the set of biometric sensor data; generating features from the processed biometric sensor data which are associated with one or more characteristic physiological event phase; determining, the set of processed biometric sensor data, or both, a confidence score for each characteristic physiological event phase of the characteristic physiological event phases indicating a presence of that phase in a data segment; determining a final confidence score indicating an occurrence of a physiological event based on a relation between the confidence scores of all the characteristic physiological events; determining a cumulative confidence score indicating an occurrence of a particular physiological event, wherein the physiological event comprises of characteristic physiological event phases; and providing a potential physiological event alert based on the cumulative confidence score.

### CROSS-REFERENCE TO RELATED APPLICATIONS

This application is the national stage entry of International Patent Application No. PCT/US2022/051365, filed on Nov. 30, 2022, and published as WO 2023/102023 A1 on Jun. 8, 2023, which claims the benefit of U.S. Provisional Patent Application No. 63/284,891, filed on Dec. 1, 2021, which are hereby incorporated by reference in their entireties.

### FIELD

This application is directed to methods and systems for physiological detection and alerting.

### BACKGROUND

Uncontrolled seizures affect up to 56% of patients with epilepsy and impose substantial physical, psychological and financial burdens. Better management requires accurate information, but this is difficult to acquire in outpatient settings. Patient reported outcomes (PRO's) have been suggested in the form of seizure diaries, but they are limited by poor adherence and post-ictal amnesia. Uncontrolled tonic-clonic seizures (TCS) greatly increase the risk of sudden unexpected death in epilepsy (SUDEP), by one estimate up to 27-fold. The risk of SUDEP can be substantially reduced by caregiver intervention, but this requires continuous monitoring and timely alerting. Indeed, multiple surveys have emphasized the need for wearables with reliable seizure monitoring to alert caregivers and provide accurate journaling of seizures, and a variety of such devices have been developed in the past decade.

To date, non-electroencephalography (non-EEG) based TCS monitoring devices have generally demonstrated acceptable sensitivities (>90%) in inpatient environments, but their false alarm rates (FAR) have been too high, in both inpatient and ambulatory environments. High FAR's risk causing alarm fatigue in both caregivers and patients, resulting in poor adherence to device monitoring and alerting. This is even more prevalent in ambulatory user environments where daily movements can frequently trigger false alarms. To date, among large scale video-electroencephalography (vEEG)-verified prospective multicenter studies (4000+ recorded hours), mean FAR's have ranged from 0.2/day-0.83/day, though there has been significant variation between algorithms, age groups and activity levels in the EMU. Thus, there remains a need to develop a seizure detection algorithm that can significantly lower FAR in both EMU and outpatient environments, while maintaining or improving current sensitivity standards.

### SUMMARY

According to examples of the present disclosure, a method for physiological event detection and alerting is disclosed. The method comprises obtaining, from one or more biometric sensors, a set of biometric sensor data from a user; generating, by one or more hardware processors, a set of processed biometric sensor data from the set of biometric sensor data; generating, by the hardware processor, a set of features from the processed biometric sensor data which are associated with one or more characteristic physiological event phase, wherein the association between the set of features and the one or more characteristic physiological event phase is stored in one or more non-transitory storage media; determining, from the set of generated features, the set of processed biometric sensor data, or both the set of generated features and the processed biometric sensor data using the one or more hardware processors, a confidence score for each characteristic physiological event phase of the one or more characteristic physiological event phase indicating a presence of that phase in a data segment; determining, from a relation between the confidence score of each of the characteristic physiological event phase using the one or more hardware processors, a final confidence score indicating an occurrence of a physiological event based on a relation between all physiological event phase confidence scores; determining, from an accumulation of final confidence scores using the one or more hardware processors, a cumulative confidence score indicating an occurrence of a particular physiological event, wherein the physiological event comprises of one or more characteristic physiological event phases; and providing, by the one or more hardware processors, a potential physiological event alert based on the cumulative confidence score.

According to examples of the present disclosure, a system for physiological event detection and alerting is disclosed. The system comprises one or more biometric sensors that capture, record, or both capture and record biosensor data from a user; one or more hardware processors; one or more non-transitory computer readable media that stores instructions, that when executed by the one or more hardware processors, perform a method of physiological detection and alerting comprising: obtaining, from the one or more biometric sensors, a set of biometric sensor data from a user; generating, by one or more hardware processors, a set of processed biometric sensor data from the set of biometric sensor data; generating, by the hardware processor, a set of features from the processed biometric sensor data which are associated with one or more characteristic physiological event phase, wherein the association between the set of features and the one or more characteristic physiological event phase is stored in one or more non-transitory storage media; determining, from the set of generated features, the set of processed biometric sensor data, or both the set of generated features and the processed biometric sensor data using the one or more hardware processors, a confidence score for each characteristic physiological event phase of the one or more characteristic physiological event phase indicating a presence of that phase in a data segment; determining, from a relation between the confidence score of each of the characteristic physiological event phase using the one or more hardware processors, a final confidence score indicating an occurrence of a physiological event based on a relation between all physiological event phase confidence scores; determining, from an accumulation of final confidence scores using the one or more hardware processors, a cumulative confidence score indicating an occurrence of a particular physiological event, wherein the physiological event comprises of one or more characteristic physiological event phases; and providing, by the one or more hardware processors, a potential physiological event alert based on the cumulative confidence score; and a user interface that provides the potential physiological event alert.

Various additional features can be included in the method and/system for physiological detection and altering including one or more of the following features described below.

The one or more biometric sensors comprise one or more of: an accelerometer, a photoplethysmography (PPG) sensor, a gyroscope, a microphone, a blood oxygenation sensor, a blood pressure sensor, a blood sugar sensor, an ocular sensor, an electrodermal activity sensor, an eye gaze sensor or tracker, a pupillometry sensor, or combinations thereof.

The one or more biometric sensors are incorporated into a wearable device comprising of a wristwatch, glasses, a cuff, a necklace, a bracelet, eyeglasses, a headset, one or more rings, or combinations thereof.

The set of preprocessed biometric data comprises filtered biometric data that is filtered for noise reduction and interpolation.

The method for physiological detection and alerting can further comprise processing the set of biometric sensor data, to produce the set of processed biometric sensor data; reducing a data set imbalance between physiological events and non-physiological events in the processed biometric sensor data by iteratively training and using one or more models to identify anomalous segments in non-physiological event biometric sensor data to produce a balanced dataset, wherein the one or more models comprise one or more anomaly detection methods; and using the balanced dataset to train one or more classifiers for each characteristic physiological event phase that produces the confidence score for each characteristic physiological event phase.

The one or more anomaly detection methods comprise one or more of: isolation forest, one class Support Vector Machines (SVM), Hidden Markov Models (HMM), Auto Encoders, Variational Auto Encoders, Cluster-based outlier detection, or combinations thereof.

Each of the characteristic physiological event phases comprises an event causing a characteristic biometric signal pattern related to a whole or a part of the physiological event, wherein the characteristic biometric signal pattern comprises one or more of: a tonic movement and/or associated physiological changes, a clonic movement and/or associated physiological changes, a post-ictal movement suppression or impairment and/or associated physiological changes, a prodromal movement and/or associated physiological changes, an early ictal movement and/or associated physiological changes, a late ictal movement and/or associated physiological changes, an ictal cry and/or associated physiological changes, a specific automatism comprising one or more of: hand shaking, shivering, paroxysmal blinking or staring, saccades, fixation, noises, movement arrest, or a specific physiological response comprising one or more of: heart rate changes or blood pressure changes.

The set of features from the processed biometric sensor data that are generated use techniques comprising one or more of: manual feature extraction, automated feature extraction, or combinations thereof. The manual feature extraction comprises one or more of: time domain feature extraction, frequency domain feature extraction, or combinations thereof. The time domain feature extraction comprises one or more of: a line crossing, a variance, a skewness, a kurtosis, or combinations thereof. The frequency domain feature extraction comprises one or more of: a fan-chirp transform, a Fourier transform, a chirp Z transform, a constant-Q Transform, a wavelet transform, or combinations thereof. The automated feature extraction comprises one or more of: one or more deep learning methods and one or more convolutional neural networks.

Confidence scores for each of the characteristic phases are calculated using classifiers comprising classical techniques comprising one or more linear models, one or more tree-based methods, one or more clustering methods, one or more probabilistic graphical models, one or more deep learning models, or combinations thereof.

The relation between the confidence scores determining the final confidence score comprises techniques of aggregating the confidence scores comprising one or more of: one or more non-temporal techniques that analyze single time points, one or more classical temporal techniques that analyze multiple time points in the past, one or more deep learning techniques, or combinations thereof. The non-temporal techniques comprise one or more of: a mean, a weighted mean, arithmetic expression of confidence scores, or combinations thereof. The temporal techniques comprise a probabilistic graphical method. The probabilistic graphical method comprises one or more Hidden Markov Models, one or more Conditional Random Fields, or both. The deep learning techniques comprise one or more of: a Recurrent Neural Network, a Long Short Term Memory Network, a Gated Recurrent Unit Network, a Temporal Convolutional Network, a Convolutional Neural Network, a Multi Layer Perceptron, or combinations thereof.

The accumulation of final confidence scores to generate the cumulative confidence score comprises one or more of: a low pass filter and a temporal modelling technique. The temporal modeling technique comprises one or more of: a Hidden Markov Model, a Conditional Random Field, a Recurrent Neural Network, a Long Short Term Memory Network, a Gated Recurrent Unit Network, a Temporal Convolutional Networks, a Convolutional Neural Networks, or combinations thereof.

The potential physiological event alert is provided on a user interface of a wearable device worn by the user. The potential physiological event alert is provided to one or more of the user, a caregiver, a healthcare provider, or a legal guardian. A physiological event is any event that causes one or more characteristic patterns that can be identified through one or more of the biometric sensors, these events comprising: epileptic seizures, syncope, psychogenic non-epileptic seizures, movement disorders, or combinations thereof.

The one or more hardware processors comprise a first processor in a first device worn on the head or face and a second processor in a second device that is worn on the wrist or another part of the body. The first device worn on the head or face is a pair of eyeglasses and the second device is a wristwatch. The physiological event comprises a neurological event, a cardiac event, or combinations thereof. The neurological event is a seizure.

### BRIEF DESCRIPTION OF THE DRAWINGS

The accompanying drawings, which are incorporated in and constitute a part of this specification, illustrate embodiments of the present teachings and together with the description, serve to explain the principles of the present teachings. In the figures:

FIG. 1A and FIG. 1B show a back perspective view and a front perspective view, respectively, of a smart watch according to examples of the present disclosure;

FIG. 2 shows a smart eyeglass according to examples of the present disclosure;

FIG. 3A and FIG. 3B show a distribution of the individual FAR rates for different patients, segmented by EMU and ambulatory environments according to examples of the present disclosure, where the circumference of the polar plots designates the hours in the day in 24 hour format and the radius shows the number of sessions that have been recorded during that time period.

FIG. 4 shows a distribution of the individual FAR rates for different patients, segmented by EMU and ambulatory environments according to examples of the present disclosure;

FIG. 5 shows a selection of tonic-clonic seizures (TCS) detected during prospective trial, centered by time of detection according to examples of the present disclosure;

FIG. 6 shows a method for physiological event detection and alerting according to examples of the present disclosure;

FIG. 7 shows a method for training data according to examples of the present disclosure;

FIG. 8 shows data from a tonic-clonic seizure according to examples of the present disclosure;

FIG. 9 shows data from a clonic phase only (not TCS) according to examples of the present disclosure;

FIG. 10 shows data from an exercise example with majority tonic phase (not TCS) according to examples of the present disclosure;

FIG. 11 shows data from a long single phase according to examples of the present disclosure;

FIG. 12 shows data from a focal to bilateral tonic-clonic seizure (FBTCS) according to examples of the present disclosure;

FIG. 13 illustrates a schematic view of a computing system according to examples of the present disclosure; and

FIG. 14 shows an exemplary method for data training and physiological event detection and alerting according to examples of the present disclosure.

### DETAILED DESCRIPTION

Reference will now be made in detail to embodiments, examples of which are illustrated in the accompanying drawings and figures. In the following detailed description, numerous specific details are set forth in order to provide a thorough understanding of the invention. However, it will be apparent to one of ordinary skill in the art that the invention may be practiced without these specific details. In other instances, well-known methods, procedures, components, circuits and networks have not been described in detail so as not to unnecessarily obscure aspects of the embodiments.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains.

Generally speaking, the present disclosure describes methods and systems for physiological detection and alerting. The methods and system can use one or more biometric sensors to collect, record, process, preprocess, or any combination thereof, biometric data from a user. The one or more biometric sensors can operate with one or more hardware processors co-located with one or more of the one or more biometric sensors or remotely located therefrom, including communicating biometric data over a networked environment to process the biometric data. The disclosed methods and systems obtain a set of biometric sensor data from a user from one or more biometric sensors. The disclosed methods and systems then generate a set of processed biometric sensor data from the set of biometric sensor data. The disclosed methods and system then generate a set of features from the processed biometric sensor data which are associated with one or more characteristic physiological event phase. The disclosed methods and systems then determine, from the set of generated features, the set of processed biometric sensor data, or both the set of generated features and the processed biometric sensor data, a confidence score for each characteristic physiological event phase of the one or more characteristic physiological event phase indicating a presence of that phase in a data segment. The disclosed methods and systems then determine, from a relation between the confidence score of each of the characteristic physiological event phase, a final confidence score indicating an occurrence of a physiological event based on a relation between all physiological event phase confidence scores. The disclosed methods and systems then determine, from an accumulation of final confidence scores, a cumulative confidence score indicating an occurrence of a particular physiological event, wherein the physiological event comprises of one or more characteristic physiological event phases. The disclosed methods and systems then provide a potential physiological event alert based on the cumulative confidence score.

Although the below discussion will deal primarily with a study that was conducted relating to seizures, this is just one example of a physiological event that can be detected and alerting using the presently disclosed methods and systems. Other neurological or cardiac events can be detected, and alerts provided based on biometrical sensor data collected from the one or more biometric sensors and analyzed by the one or more processors operating the disclosed methods.

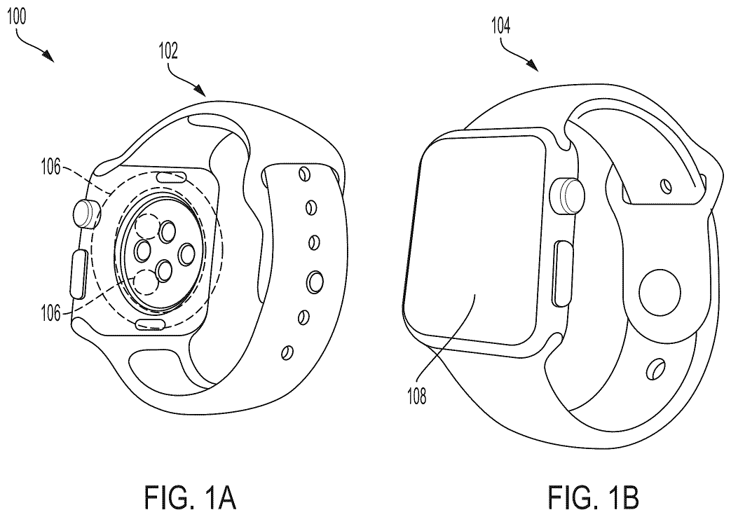

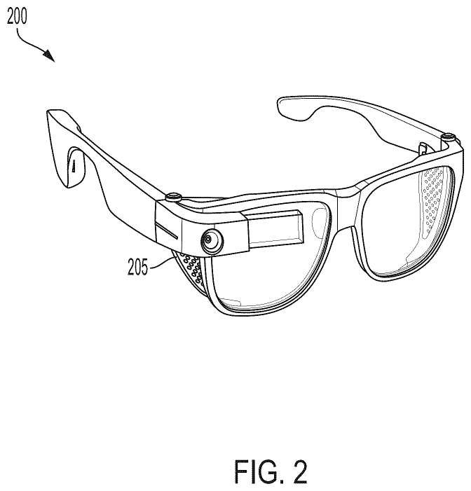

FIG. 1A and FIG. 1B show a back perspective view 102 and a front perspective view 104 of a smart watch 100 according to examples of the present disclosure. On the back of the smart watch 100, which is in contact with the skin of the user, one or more biometric sensors 106, such as one or more PPG sensors. The front of the smart watch 100 includes a user interface 108 to provide personalized alerts to the user. The smart watch 100 can include additional sensors such as, but not limited to an accelerometer, a gyroscope, a microphone, a blood oxygenation sensor, a blood pressure sensor, a blood sugar sensor, an electrodermal activity sensor, combinations thereof. FIG. 2 shows smart eyeglasses with sensor 205 according to examples of the present disclosure. Sensor 205 can include, but are not limited to, an ocular sensor, an eye gaze sensor or tracker, a pupillometry sensor, or combinations thereof. The smart eyeglasses can be used alone or in combination with smart watch 100 to provide biometric sensor data that can be used for physiological event detection and alerting.

For example, monitoring for tonic-clonic seizures (TCS) is important for enhancing safety, promoting independence, and avoiding sudden unexpected deaths in epilepsy (SUDEP). Any system for TCS monitoring should be highly sensitive, present a low false alarm rate (FAR) and provide alerts to caregivers with a low latency across use in both inpatient and ambulatory environments. Ideally, these devices should also be non-invasive, multifunctional and avoid stigma. The preliminary study described below shows the performance characteristics of a seizure monitoring application that uses the disclosed methods and implemented on a consumer smart watch, that was tested in both inpatient and ambulatory environments. In this study, TCS's encompassed all major tonic-clonic seizure types as defined by the ILAE, including generalized tonic-clonic seizures (GTCS), focal to bilateral tonic-clonic seizures (FBTCS), unknown onset tonic-clonic seizures (UTCS) and myoclonic-tonic-clonic seizures (MTCS).

In this study, data was initially collected from 340 patients in 4 Epilepsy Monitoring Units (EMUs), and 21 ambulatory users (13 outpatients with epilepsy, 8 normal controls without epilepsy). Accelerometer (ACM) and heart rate signals were recorded with the application developed for smart watch. Other biometric data can also be collected including, but not limited to, electromyogram (EMG), photoplethysmography (PPG), and electrodermal activity (EDA). Seizures in the EMUs were validated with video-electroencephalography (vEEG), while ambulatory user seizure events were self-reported and not included in the training set as they were difficult to validate. This yielded a dataset of 20,388 hours including 79 TCS (58 EMU patients), and 5,642 seizure-free hours from ambulatory users (outpatients and normal controls). This data was used to train a novel classifier in an offline environment that was subsequently implemented on the smart watch, as part of the seizure monitoring application. Prospective testing was performed on 85 unique EMU patients and 15 ambulatory users (9 with outpatients, 6 normal controls). EMU patients were blinded to seizure detections, and seizures were validated with vEEG. Ambulatory users were unblinded to seizure detections, and seizures were self-reported or retrospectively identified through independent bio-signal analysis. The testing dataset was 4,279 hours in the EMU with 19 seizures (15 patients) and 6,735 hours in outpatients with 10 self-reported seizures (3 patients). Prospective testing resulted in a positive percent agreement (PPA) of 100%, an FAR of 0.05 per day in the EMU (positive predictive value, PPV, of 68%) and 0.13 per day in ambulatory users (PPV of 22%). A single outpatient was responsible for 8 of 31 total false alarms. The FAR for all other ambulatory users excluding this outpatient was 0.10 per day. Mean detection latency was 37.38 s (stdev=13.24 s) in the EMU and 32.07 s (stdev=10.22 s) in ambulatory users.

In the study, all biosensor data was collected using a smart watch and a paired smart phone. Each smart watch was installed with the application called EpiWatch that enabled collection from the watch's built-in 3-axis accelerometer (ACM; 50 Hz sampling frequency) and PPG (0.2 Hz sampling frequency) sensors. The body of the watch was placed on the dorsal side of the wrist, ensuring a snug fit between the PPG sensor and skin to prevent data loss. At all EMU sites, the watch was placed on the arm where motor manifestations were more apparent, if this was known.

When the watch was actively monitoring, the EpiWatch application de-identified, encrypted, and stored watch sensor data on a cloud-based backend for further analysis and algorithm development. During the study, a TCS detection algorithm was implemented in the EpiWatch application that continuously monitored the sensor data to provide TCS detection and alerting. All alerts and events from the monitor were also stored on the cloud-based backend. For redundancy, all alerts were also stored on the watch and could be retrieved manually or were automatically stored on the cloud-based backend in the event that connectivity was temporarily interrupted.

The EMU training dataset consisted of 20,388 hours of data recorded across four sites; Johns Hopkins Hospital Adult EMU (JHA), Le Bonheur Children's Hospital Pediatric EMU (LBH), Ruber International Hospital Adult EMU (RBI), and the University of Maryland Medical Center Adult EMU (UMD). There were a total of 340 unique users and 79 seizures (from 58 users). Each seizure was validated by two board-certified clinical neurologists using vEEG and classified as either a TCS or not.

The ambulatory user (AMB) dataset consisted of 5,462 hours in total, recorded across outpatient users with epilepsy (OUT) and normal control users without epilepsy (NC). The outpatient set consisted of PWE testing the algorithm during their normal activities outside the hospital. Seizures from outpatient users were not included in the training dataset due to the inherent limitations of validating seizure type and occurrence without vEEG. These seizure segments were discovered using Patient Reported Outcomes (PROs), automated motion detection and visual analysis of time series segments. Despite best efforts, there is a possibility that some TC seizures still existed in this dataset.

The normal control dataset consisted of day-to-day user activity obtained to estimate FAR during activities that have conventionally caused false alarms in monitoring devices. These activities include brushing teeth, exercise, washing dishes, drumming, dancing, etc. A breakdown of the training dataset is provided in Table 1.

Table 1. Data used for monitor training, consisting of hours recorded, number of users recorded from, number of total tonic-clonic seizures (TCS) and the number of users that experienced at least one seizure. The ambulatory (AMB) setting is segmented into outpatient (OUT) and non-control user (NC; ambulatory users without epilepsy) sites. The EMU setting is segmented into four separate hospital EMUs: Johns Hopkins Adult (JHA), University of Maryland (UMD), Ruber International (RBI), La Bonheur Children's (LBH).

| | | | | Users | Seizure |
|---|---|---|---|---|---|
| Setting | Site | Hours | Total | TC Seizures | Users |
| AMB | OUT | 3,497.82 | 8 | — | — |
| | NC | 1,964.38 | 13 | — | — |
| | Total | 5,462.20 | 21 | — | — |
| EMU | JHA | 12,143.05 | 172 | 61 | 45 |
| | RBI | 12,87.25 | 36 | 3 | 3 |
| | LBH | 6,254.27 | 123 | 15 | 10 |
| | UMD | 7,04.12 | 9 | 0 | 0 |
| | Total | 20,388.69 | 340 | 79 | 58 |

Real-time out-of-sample performance testing of the detection algorithm was performed across four epilepsy monitoring unit (EMU) sites: adult and pediatric EMUs in the Johns Hopkins Hospital (6 beds JHA and 4 beds JHP, respectively), the pediatric EMU of Le Bonheur Hospital (10 beds LBH), and the adult EMU of Ruber International Hospital (3 beds RBI). The patients that participated during the testing phase were different from patients that contributed data for training the seizure detection algorithm. The algorithm was not altered during the entire testing period. To prevent patient/caregiver bias, all detections were silent with alerts being sent directly to a central coordinator, with the corresponding timestamps logged on the cloud-based backend. EMU seizures were validated by two board-certified clinical neurologists using video-EEG. The video-EEG data was examined either at the EMU site hospital, or at Johns Hopkins Hospital after being sent via encrypted hard-drive. To ensure continuous tracking, nursing staff at the EMU sites were asked to charge watches twice a day, once around 7 am, and again around 7 μm, or use multiple watches for a single patient if watches were available. These times were chosen to reduce the risk of missing a seizure during charging periods. Charging watches would automatically discontinue tracking if it was not done so manually, to prevent artificially increasing the overall recording period.

The detection algorithm was also tested among outpatients to obtain performance in a real-life environment. Users either already owned or were provided a smart watch and paired smart phone, and asked to download the application through an application store. The application has built in e-consenting for subjects and caregivers-once the subject or legally authorized representative provided consent they were invited to participate in testing of the algorithm in an ambulatory setting. Subjects and caretakers were warned not to rely on EpiWatch detection as a stand-alone method to get help with their seizures. The detection algorithm was not altered for outpatients during the entire testing period. As most outpatient users were using the application in a realistic manner, alerting functionality was enabled for this set of users, with alerts being sent to their chosen caregiver.

Outpatient seizures were validated primarily through communication with patients/caregivers, PROs (see above), and manual bio-signal analysis. Some subjects with video monitoring in their houses provided video evidence of convulsing behaviors. Nurses also conducted interviews with caregivers of patients that witnessed or arrived shortly after a seizure had occurred. To mitigate the risk of false negatives from potentially unreported seizures, follow up communications were performed with the outpatients and/or caregivers in addition to automated motion detection and manual bio-signal analysis. While unlikely, false negatives may still have occurred during the prospective study in outpatients.

For EMU training and the prospective test set, there were a set of exclusion criteria developed to classify a specific recorded segment as a valid TCS for this preliminary study. Seizures that met any of the criteria were excluded from the final metrics.
E1. EEG data was not present for the time period that the seizure occurred
E2. The patient was not on video for the time period that the seizure occurred
E3. The Apple Watch with the running detection algorithm was not on the patient's wrist and actively monitoring.
E4. At least 2 board certified epileptologists did not come to a consensus of TCS classification after video-EEG review according to the ILAE classification system.
E5 Motor manifestations of TCS did not occur in the limb being monitored by the watch, e.g. unilateral TCS
E6. Limb motion was excessively damped by any external entity (caregiver/nurse/physician). Note that in this case, TCS monitoring would not be necessary.

Additionally, during the prospective study, as soon as the device logged a detection, it was counted as a detection in the results, unless it could be verified via video-EEG and collected data, that the watch was not on the patient and tracking at the time of detection.

During the study, each vEEG-validated seizure and/or algorithm detection was treated as an event classified as either a True Positive (TP; segment was a TCS and detected by the algorithm), a False Positive (FP; segment was not a TCS but was detected by the algorithm, aka false alarm), or a False Negative (FN; segment was a TCS but was not detected by the algorithm). To enable comparison with other papers in the field, similar metrics have been used, namely Sensitivity/Positive Percent Agreement (PPA) with 95% Confidence Interval (CI), Precision/Positive Predictive Value (PPV) with 95% CI, and False Alarm Rate (FAR) with 95% CI and Latency.

PPA describes how effective the detector is at detecting TCS's. Due to the potential negative consequences of false negatives, the PPA value is ideally 100%, though in practice this would be impossible without generating an unacceptably high false alarm rate. PPV describes how likely a given detection is a true positive. For detectors that generate a lot of false positives (false alarms), the PPV value will be low (close to 0). As PPA and PPV are both binomial proportion metrics (and thus between 0 and 1), a Wilson Score with continuity correction was used to calculate the 95% CI. The Wilson Score has been shown to be the most accurate and robust among known binomial proportion confidence interval calculation methods. We used continuity correction as it provides a slightly more conservative CI estimate as compared to no correction.

FAR is defined as the number of false alarms (FAs; alternatively false positives FPs) that occur per 24 hour period. There is no universal standard for how data should be split to calculate FAR, so we chose to use metrics of micro FAR and macro FAR. Micro FAR is calculated as the total number of false alarms across all users, divided by the total number of recorded hours across all users. Macro FAR is calculated as the average of the FAR calculated for each user individually. Micro FAR can be thought of as the weighted version of the macro FAR, weighted by the proportional number of hours recorded for a specific user. In general, most papers have reported micro FAR in their metrics.

As FAR is not a binomial proportion, a non-parametric bootstrap method is used to approximate its 95% confidence interval. Sampling with replacement was performed at the patient-level to account for intra-patient variability, with 10,000 separate samples drawn for the FAR which is large enough for our chosen a value of 0.05. The resulting distribution is approximately normal. To find the confidence intervals, the 2.5th and 97.5th percentiles of the samples were chosen, to the nearest sample value. The method varied slightly between micro and macro-FAR, being calculated separately for each of the 10,000 samples. However, bootstrapping was only performed once, with the same sampled data being used to estimate micro and macro-FAR CI's.

Latency is defined as the time it takes for a detection to occur after seizure onset. It is a difficult metric to consolidate because setting the seizure start time is somewhat subjective. We selected the start time as the onset of motor manifestations of the tonic clonic phase as evident on video and recorded ACM data. The start times for all seizures in the study were determined by at least one board-certified epileptologist. The latency mean and standard deviation values was measured for inpatients and outpatients.

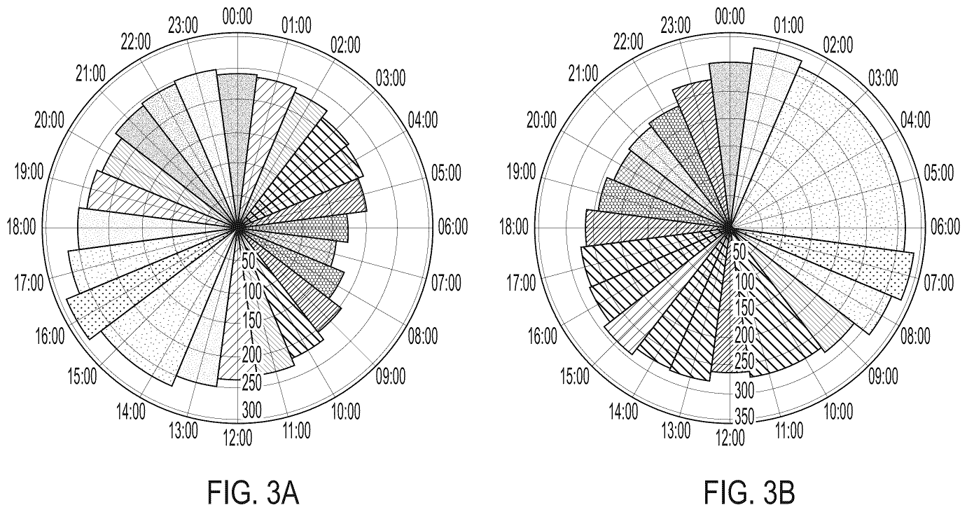

FIG. 3A and FIG. 3B show a distribution of the individual FAR rates for different patients, segmented by EMU and ambulatory environments according to examples of the present disclosure, where the circumference of the polar plots designates the hours in the day in 24 hour format and the radius shows the number of sessions that have been recorded during that time period.

Over the course of 206 days (4,279.88 hours), 85 new out-of-sample (OOS; not previously included in the training data) patients diagnosed with epilepsy were enrolled for vEEG monitoring at 4 EMU sites; JHA (29 patients), LBH (17 patients), RBI (37 patients) and Johns Hopkins Hospital Pediatric EMU (JHP; 2 patients). These patients were monitored by using an smart watch running the EpiWatch research app with real-time monitoring for TCS. Across the 4 EMUs there were 28 detections, and 19 TCS confirmed through vEEG, coming from 15 unique patients. A breakdown of EMU session coverage in FIG. 3A and FIG. 3B indicates that on average, the smart watches were approximately charged twice a day, once at 7 am and once at 7 pm.

Ambulatory user testing was performed for over 6,735.03 hours on 15 total users (6 out-of-sample users), from two groups: outpatient users (9 users, 5 out-of-sample) and normal control users (6 users, 1 out-of-sample). The ambulatory users were asked to use the algorithm as often as they could, aiming for 24 hr coverage, though timings varied as different users charged the devices at different times throughout the day. A breakdown of ambulatory user session coverage is provided in FIG. 3A and FIG. 3B.

Table 2. Total hours recorded during prospective testing alongside total users and out-of-sample (OOS) users. Data is segmented by ambulatory (AMB) and EMU settings with their respective sites. The ambulatory setting is segmented into outpatient (OUT) and non-control user (NC; ambulatory users without epilepsy) sites. The EMU setting is segmented into four separate hospital EMUs: Johns Hopkins Adult (JHA), Johns Hopkins Pediatric (JHP), Ruber International (RBI), La Bonheur Children's (LBH).

| Setting | Site | Hours | Total Users | OOS Users |
|---|---|---|---|---|
| AMB | OUT | 5,137.90 | 9 | 5 |
| | NC | 1,597.13 | 6 | 1 |
| | Total | 6,735.03 | 15 | 6 |
| EMU | JHA | 1,603.63 | 29 | 29 |
| | RBI | 880.18 | 17 | 17 |
| | LBH | 1,724.4 | 37 | 37 |
| | JHP | 71.67 | 2 | 2 |
| | Total | 4,279.88 | 85 | 85 |

For the evaluation of performance for continuous monitoring, it was determined whether the performance metrics of the detection algorithm were not biased by uneven amounts of data collected at any particular time of day. To this end, polar plots of cumulative recording periods are shown in FIG. 3A and FIG. 3B to confirm that time periods throughout the day were monitored approximately uniformly. Notably, the decrease in recorded times during the periods 06:00-08:00 and 19:00-21:00 for EMU users, and 11:00-13:00 and 19:00-21:00 for outpatients indicates periods when Apple Watches were being charged. Ambulatory users had a tendency towards night-time tracking sessions (especially 01:00-09:00), while EMUs had a tendency towards day-time tracking sessions (especially 13:00-17:00).

FIG. 3A and FIG. 3B show the distributions over time of recorded time periods during tracking sessions. FIG. 3A shows the EMU tracking distribution, and FIG. 3B shows the ambulatory user tracking distribution. The circumference of the polar plots designates the hours in the day in 24 hour format. The radius shows the number of sessions that have been recorded during that time period.

A total of 29 seizures occurred during the 10,990.81 hours of monitoring across EMUs (19 TCS) and outpatients (10 TCS). For EMUs in particular, the seizure distribution was relatively uniform, with 15 unique patients having seizures. In the outpatients, only 3 users had seizures, with one user having 8 TCS. The number of hours recorded between adult and pediatric patients were relatively similar, with 2,483 hours being recorded in adult EMUs, and 1,796 hours being recorded in pediatric EMUs.

For both outpatients and EMU patients, all seizures were detected by the monitoring application, leading to a PPA of 100% (1.0). Due to having only 10 seizures in the outpatient dataset and using the more conservative continuity corrected Wilson method, the 95% CI was wide, with a lower bound of 0.66. The EMU PPA 95% CI was far narrower due to the increased sample size, with a lower bound of 0.79. Recent FDA clearances for devices in this class have designated a minimum required PPA CI lower bound of 0.7 [5] (though it is unclear what method was used to calculate the CIs).

At the operating point chosen for this study, there were only 9 false alarms in 4,280 hours of tracking across all four EMUs. This translated to a micro FAR of 0.05/day, with a 95% CI of [0.02, 0.08]. The micro FAR and macro FAR seemed to align well for all the EMUs, meaning no single patient was affecting the FAR calculation in a significant manner. The exception was RBI, where a macro FAR of 0.22 suggested that a minority of patients had short recording periods with a high FAR. This was indeed the case, with a single RBI patient having 1 FA in a total recording period of 7.9 hours, resulting in an FAR of 3.05/day for that patient. With only 19 seizures occurring in the 4,280 total hours recorded in the EMU, the PPV was 0.68 [0.48, 0.83], meaning approximately two TP for every one FA. Interestingly, comparing the macro FAR for pediatric patients (LBH and JHP) of 0.03/day to the macro FAR for adult patients (JHA and RBI) of 0.07/day, patients in adult EMUs appeared to be causing more FAs than patients in the pediatric EMUs.

For the ambulatory user dataset, there were 36 false alarms in 6,735 hours of recording, leading to a micro FAR of 0.13/day [0.08, 0.24]. As expected, ambulatory users had a higher FAR than the inpatient users. In fact, one of the outpatient users received 8 false alarms over 24 hours of recording, and consequently requested to stop using the device. This suggests that there were activities for which the algorithm was still not specific enough, likely because the distribution of all possible ambulatory motion was not adequately represented within the training set. Due to the extremely high FAR for this user, we included the adjusted metrics (shown by the italicized metrics) had this user been classified as an outlier in Table 3. While the micro FAR only decreased by 0.03/day (0.14/day to 0.11/day), the macro FAR decreased from 0.97 to 0.09/day, confirming the large contribution to the FAR from the single beta tester. The PPV of 0.22 [0.11, 0.37] for the outpatient dataset resulted in detector performance of approximately one TP for every three FAs.

Table 3. Performance characteristics of the seizure monitoring application segmented by ambulatory and EMU settings, and their respective sites. The ambulatory setting is segmented into outpatient (OUT) and non-control user (NC; ambulatory users without epilepsy) sites. The EMU setting is segmented into four separate hospital EMUs: Johns Hopkins Adult (JHA), Johns Hopkins Pediatric (JHP), Ruber International (RBI), La Bonheur Children's (LBH).

| | | | Users | | | | | | | | | | | Latency |
|---|---|---|---|---|---|---|---|---|---|---|---|---|---|---|
| Setting | Site | Hours | (w/TCS) | | TCS | Det. | TP | FP | FN | FPR Micro | FPR Macro | PPA | PPV | (Std) |
| AMB | OUT | 5,137.90 | 9 | (3) | 10 | 41 | 10 | 31 | 0 | 0.14 | 0.97 | 1.0 | 0.24 | — |
| | | | | | | | | | | | | | [0.13, 0.41] | |
| | | 5,113.80 | 8 | (3) | | 33 | | 23 | | 0.11 | 0.09 | | 0.30 | |
| | | | | | | | | | | | | | [0.16, 0.49] | |
| | NC | 1,597.13 | 6 | (—) | — | 5 | — | 5 | — | 0.08 | 0.02 | — | — | — |
| | | 6,735.03 | 15 | (3) | 10 | 46 | 10 | 36 | 0 | 0.13 | 0.50 | 1.0 | 0.22 | 37.38 (13.24) |
| | | | | | | | | | | [0.08, 0.24] | [0.03, 1.65] | [0.66, 1.0] | [0.11, 0.37] | |
| | | 6,710.93 | 14 | (3) | | 38 | | 28 | | 0.10 | 0.06 | | 0.26 | |
| | | | | | | | | | | [0.07, 0.14] | [0.02. 0.11] | | [0.14, 0.43] | |
| EMU | JHA | 1,603.63 | 29 | (7) | 10 | 15 | 10 | 5 | 0 | 0.07 | 0.06 | 1.0 | 0.67 | — |
| | RBI | 880.18 | 17 | (3) | 3 | 5 | 3 | 2 | 0 | 0.05 | 0.22 | 1.0 | 0.60 | — |
| | LBH | 1,724.4 | 37 | (4) | 5 | 7 | 5 | 2 | 0 | 0.03 | 0.02 | 1.0 | 0.71 | — |
| | JHP | 71.67 | 2 | (1) | 1 | 1 | 1 | 0 | 0 | 0.00 | 0.00 | 1.0 | 1.00 | — |
| | | 4,279.88 | 85 | (15) | 19 | 28 | 19 | 9 | 0 | 0.05 | 0.08 | 1.0 | 0.68 | 32.07 (10.22) |
| | | | | | | | | | | [0.02, 0.08] | [0.02, 0.16] | [0.79, 1.0] | [0.48, 0.83] | |

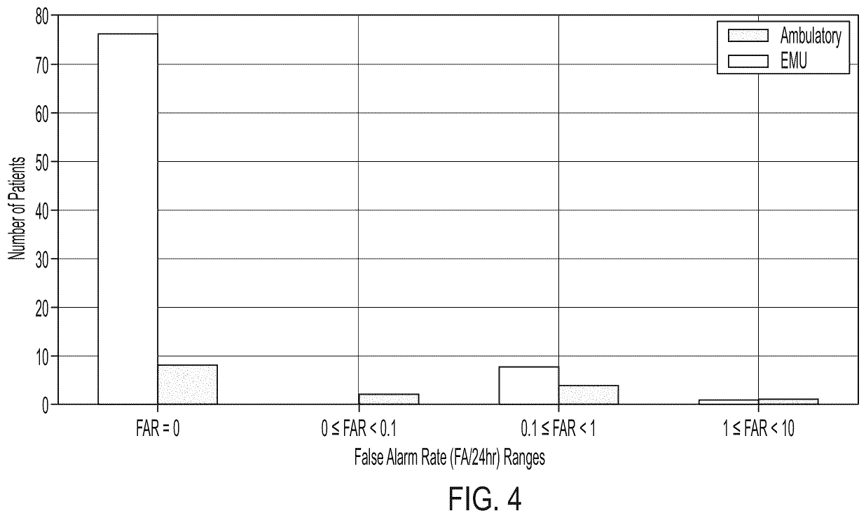

FIG. 4 shows a distribution of the individual FAR rates for different patients, segmented by EMU and ambulatory environments according to examples of the present disclosure.

Latency testing was performed for all the captured seizure data by finding the difference between the behavioral onset of the seizure and the time of detection. These latencies used the timestamps for detections captured directly on the backend, which were logged whenever the algorithm generated a detection in real-time during testing. The resulting latencies had a mean and standard deviation of 37.38 s (13.24 s) for outpatients, and 32.07 s (10.22 s) for EMU patients. The range of latencies was [22 s-67 s] for outpatients, and [20 s-57 s] for EMU patients. A selection of ACC signals during seizures, offset from the time of detection, is shown in FIG. 4 to illustrate the latencies for seizure detection, relative to seizure onset.

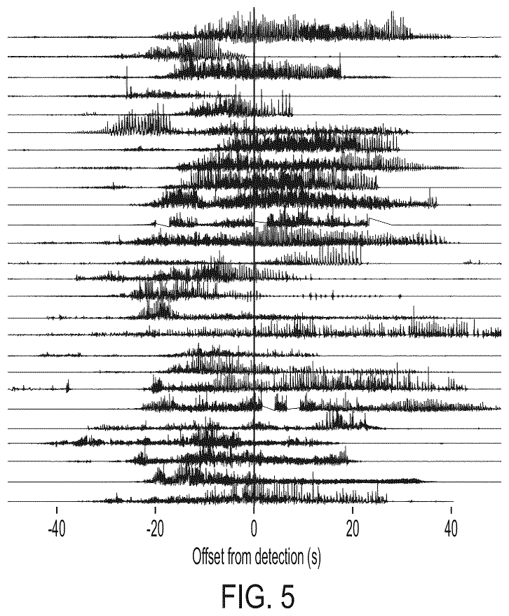

FIG. 5 shows a selection of tonic-clonic seizures (TCS) detected during prospecitve trial, centered by time of detection according to examples of the present disclosure.

To facilitate SUDEP prevention and enhance overall control of epilepsy in patients, TCS monitoring should have a very high PPA and a low enough latency such that caregivers are able to administer aid in time and prevent any potentially life-threatening outcomes. To promote consistent and continuous use of the monitor in the daily lives of people with epilepsy, it is equally important that monitoring does not generate frequent false alarms that result in alarm fatigue in the users and caregivers. Surveys of people with epilepsy and their caregivers have shown interest in non-EEG based, standalone, multi-functional devices that can be worn without risk of stigma. With these considerations, we aimed to evaluate the performance of TCS monitoring in EMU and outpatient environments using a custom application (EpiWatch) developed for a smart watch.

The general metrics used in evaluation of seizure detection devices are PPA, FAR, PPV and latency. These are also the metrics that the FDA commonly evaluates when determining whether a specific device can be cleared for seizure detection. The algorithm showed a perfect PPA of 1.0 for both outpatients (10 TCS) and inpatients (19 TCS). This is generally similar to the performance of other commercially available seizure detection devices, though many of the larger studies have been able to test algorithms against a larger sample size of seizures. Due to the nature of SUDEP, it is not only important to detect seizures, but to detect them quickly, with SUDEP being preventable if aid is administered <1 min following seizure termination. If it is assumed that most TCS are around 70 s in length, detections that occur within 50 s of seizure onset should provide enough time for caregivers to be alerted and administer aid. The current algorithm has a mean latency of 32 s (stdev=10.22 s) for EMU seizures, and 37 s (stdev=13.24 s) for outpatient seizures, which is similar to the performance of FDA cleared seizure detectors currently available on the market and is fast enough to alert caregivers and prevent SUDEP. There are still seizures that have longer latencies, with the slowest detection being 67 s for EMU seizures, and 57 s for outpatient seizures. It may be possible to tweak the operating characteristics of the algorithm and reduce the latency even further, but this may also come at the expense of a higher FAR and lower adherence.

When in general use, it is important that both caregivers and patients respond appropriately to alarms. A seizure detector that generates too many false alarms will cause alarm fatigue, causing lack of trust in the device efficacy and reducing adherence to monitoring. The algorithm tested in this study showed an excellent FAR of 0.05/day in the EMU, and 0.13/day in outpatients, translating to roughly 1 false alarm every 20 days, and 1 false alarm every 7.5 days respectively. The FAR rate was unsurprisingly higher in outpatients, with most false alarms reported as being caused by activities like mowing the lawn, starting an outdoor motor, and drumming. There was also a difference between adult (0.07/day) and pediatric (0.03/day) EMU macro FARs. This was probably caused by the training distribution being well tuned to pediatric patient behavior, even though it may be more active than adult patient behavior.

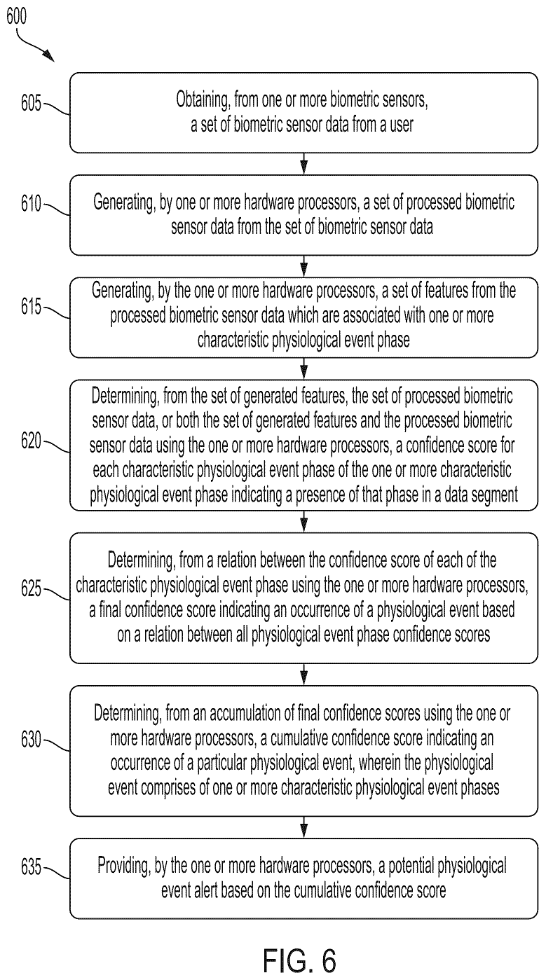

FIG. 6 shows a method for physiological event detection and alerting 600, according to examples of the present disclosure. The method 600 comprises obtaining, from one or more biometric sensors, a set of biometric sensor data from a user, as in 605. In some examples, the one or more biometric sensors comprise one or more of: an accelerometer, a photoplethysmography (PPG) sensor, a gyroscope, a microphone, a blood oxygenation sensor, a blood pressure sensor, a blood sugar sensor, an ocular sensor, an electrodermal activity sensor, an eye gaze sensor or tracker, a pupillometry sensor, or combinations thereof. In some examples, the one or more biometric sensors are incorporated into a wearable device comprising of a wristwatch, a cuff, a necklace, a bracelet, eyeglasses, a headset, one or more rings, or combinations thereof.

The method 600 further comprises generating, by one or more hardware processors, a set of processed biometric sensor data from the set of biometric sensor data, as in 610. In some examples, the set of features from the processed biometric sensor data that are generated use techniques comprising one or more of: manual feature extraction, automated feature extraction, or combinations thereof. In some examples, the manual feature extraction comprises one or more of: time domain feature extraction, frequency domain feature extraction, or combinations thereof. In some examples, the time domain feature extraction comprises one or more of: a line crossing, a variance, a skewness, a kurtosis, or combinations thereof. In some examples, the frequency domain feature extraction comprises one or more of: a fan-chirp transform, a Fourier transform, a chirp Z transform, a constant-Q Transform, a wavelet transform, or combinations thereof. In some examples, the automated feature extraction comprises one or more of: one or more deep learning methods and one or more convolutional neural networks. In some examples, the one or more hardware processors comprise a first processor in a first device worn on the head or face and a second processor in a second device that is worn on the wrist or another part of the body. In some examples, the first device worn on the head or face is a pair of eyeglasses and the second device is a wristwatch.

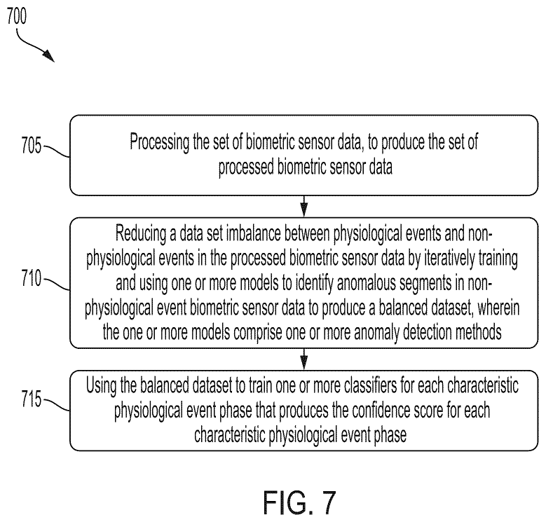

In some examples, the set of preprocessed biometric data comprises filtered biometric data that is filtered for noise reduction and interpolation. In some examples, the set of processed biometric sensor data can be processed as shown in FIG. 7 as follows. In some examples, the method 700 can further comprise processing the set of biometric sensor data, to produce the set of processed biometric sensor data, as in 705. The method 700 can also further comprise reducing a data set imbalance between physiological events and non-physiological events in the processed biometric sensor data by iteratively training and using one or more models to identify anomalous segments in non-physiological event biometric sensor data to produce a balanced dataset, wherein the one or more models comprise one or more anomaly detection methods, as in 710. The method 700 can also further comprise using the balanced dataset to train one or more classifiers for each characteristic physiological event phase that produces the confidence score for each characteristic physiological event phase, as in 715.

In some examples, the one or more anomaly detection methods comprise one or more of: isolation forest, one class Support Vector Machines (SVM), Hidden Markov Models (HMM), Auto Encoders, Variational Auto Encoders, Cluster-based outlier detection, or combinations thereof.

Returning to FIG. 6, the method 600 further comprises generating, by the hardware processor, a set of features from the processed biometric sensor data which are associated with one or more characteristic physiological event phase, as in 615. In some examples, each of the characteristic physiological event phases comprises an event causing a characteristic biometric signal pattern related to a whole or a part of the physiological event, wherein the characteristic biometric signal pattern comprises one or more of: a tonic movement and/or associated physiological changes, a clonic movement and/or associated physiological changes, a post-ictal movement suppression or impairment and/or associated physiological changes, a prodromal movement and/or associated physiological changes, an early ictal movement and/or associated physiological changes, a late ictal movement and/or associated physiological changes, an ictal cry and/or associated physiological changes, a specific automatism comprising one or more of: hand shaking, shivering, paroxysmal blinking or staring, saccades, fixation, noises, movement arrest, or a specific physiological response comprising one or more of: heart rate changes or blood pressure changes.

The method 600 further comprises determining, from the set of generated features, the set of processed biometric sensor data, or both the set of generated features and the processed biometric sensor data using the one or more hardware processors, a confidence score for each characteristic physiological event phase of the one or more characteristic physiological event phase indicating a presence of that phase in a data segment, as in 620. In some examples, confidence scores for each of the characteristic phases are calculated using classifiers comprising classical techniques comprising one or more linear models, one or more tree-based methods, one or more clustering methods, one or more probabilistic graphical models, one or more deep learning models, or combinations thereof.

The method 600 further comprises determining, from a relation between the confidence score of each of the characteristic physiological event phase using the one or more hardware processors, a final confidence score indicating an occurrence of a physiological event based on a relation between all physiological event phase confidence scores, as in 625. In some examples, the relation between the confidence scores determining the final confidence score comprises techniques of aggregating the confidence scores comprising one or more of: one or more non-temporal techniques that analyze single time points, one or more classical temporal techniques that analyze multiple time points in the past, one or more deep learning techniques, or combinations thereof. In some examples, the non-temporal techniques comprise one or more of: a mean, a weighted mean, arithmetic expression of confidence scores, or combinations thereof. In some examples, the temporal techniques comprise a probabilistic graphical method. In some examples, the probabilistic graphical method comprises one or more Hidden Markov Models, one or more Conditional Random Fields, or both. In some examples, the deep learning techniques comprise one or more of: a Recurrent Neural Network, a Long Short Term Memory Network, a Gated Recurrent Unit Network, a Temporal Convolutional Network, a Convolutional Neural Network, a Multi Layer Perceptron, or combinations thereof.

The method 600 further comprises determining, from an accumulation of final confidence scores using the one or more hardware processors, a cumulative confidence score indicating an occurrence of a particular physiological event, wherein the physiological event comprises of one or more characteristic physiological event phases, as in 630. In some examples, the accumulation of final confidence scores to generate the cumulative confidence score comprises one or more of: a low pass filter and a temporal modelling technique. In some examples, the temporal modeling technique comprises one or more of: a Hidden Markov Model, a Conditional Random Field, a Recurrent Neural Network, a Long Short Term Memory Network, a Gated Recurrent Unit Network, a Temporal Convolutional Networks, a Convolutional Neural Networks, or combinations thereof.

The method 600 further comprises providing, by the one or more hardware processors, a potential physiological event alert based on the cumulative confidence score, as in 635. In some examples, the potential physiological event alert is provided on a user interface of a wearable device worn by the user. In some examples, the potential physiological event alert is provided to one or more of the user, a caregiver, a healthcare provider, or a legal guardian. A physiological event is any event that causes one or more characteristic patterns that can be identified through one or more of the biometric sensors, these events comprising: epileptic seizures, syncope, psychogenic non-epileptic seizures, movement disorders, or combinations thereof. In some examples, the physiological event comprises a neurological event, a cardiac event, or combinations thereof. In some examples, the neurological event is a seizure.

Although FIG. 6 and FIG. 7 show example blocks of process 600 and 700, in some implementations, process 600 and 700 may include additional blocks, fewer blocks, different blocks, or differently arranged blocks than those depicted in FIG. 6 and FIG. 7, respectively. Additionally, or alternatively, two or more of the blocks of process 600 and 700 may be performed in parallel.

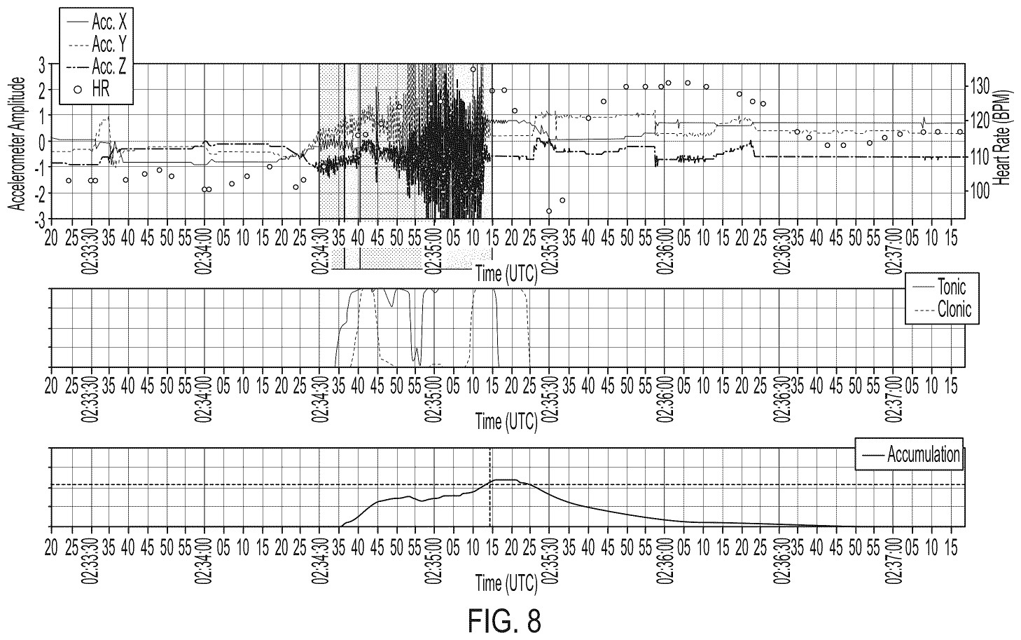

FIG. 8 shows data from a tonic-clonic seizure according to examples of the present disclosure. The top figure is the X, Y, Z accelerometer trace and heart rate (BPM), as calculated through the PPG. The middle figure is model outputs for the tonic and clonic classifiers. Note the overlap, and that there can be multiple clonic periods inside a TCS. Also please note that the classifier is lagged because of how the classifier is trained, the length of the window of data used for classification (20 seconds) and the fact that classifications are made causally. The dense dot texture region defines the tonic phase, and the sparse dot texture region defines the clonic phase(s). The bottom figure is the accumulation filter with a preset alert threshold. The threshold can be flexibly set, with a lower threshold possibly resulting in more false positive alerts (meaning that a seizure did not happen, but the method classifying the user's activity as being a seizure). For example, the threshold can be set to about 0.5 to about 0.7.

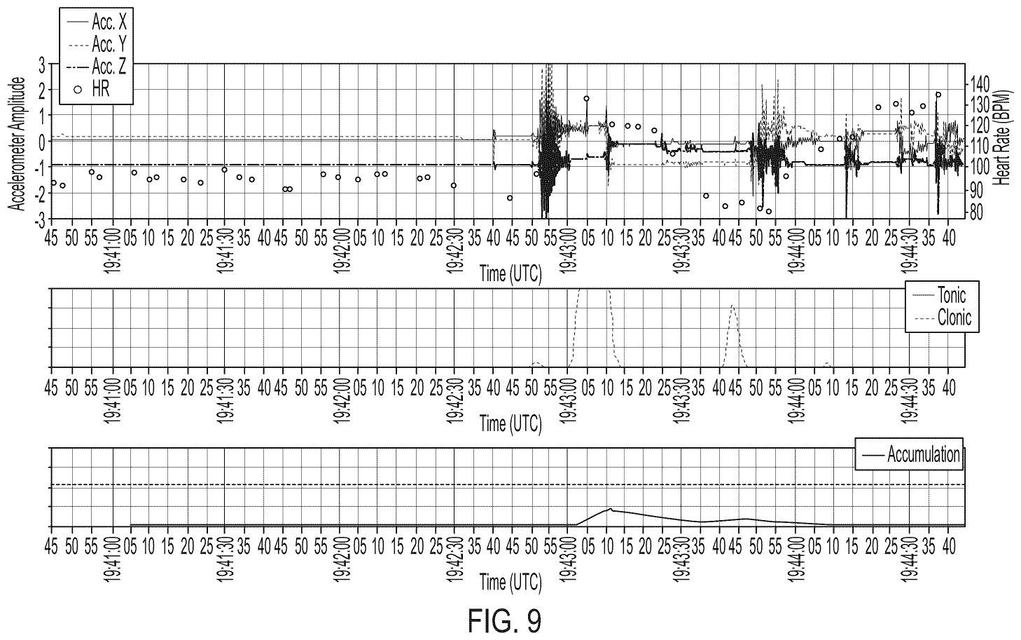

FIG. 9 shows data from a clonic phase only (not TCS) according to examples of the present disclosure. The top figure is the X, Y, Z accelerometer trace and heart rate (BPM), as calculated through the PPG. The middle figure is model outputs for the tonic and clonic classifiers. The bottom figure is the accumulation filter. Note the overlap, and that there can be multiple clonic periods inside a TCS. Also please note that the classifier is lagged because of how the classifier is trained, the length of the window of data used for classification (20 seconds) and the fact that classifications are made causally. Note that there is no activation at all from the tonic phase classifier.

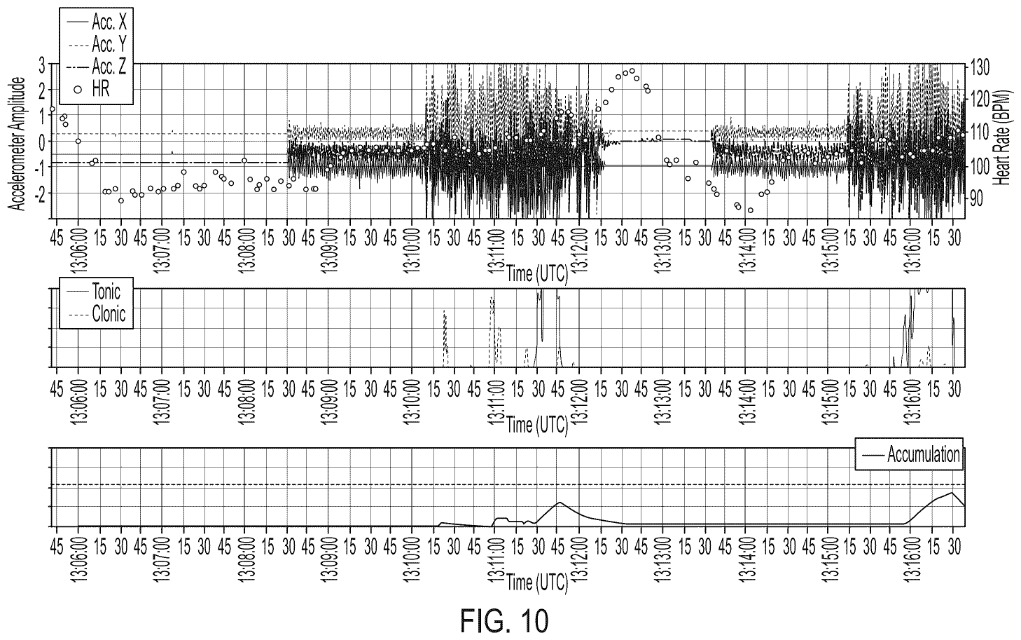

FIG. 10 shows data from an exercise example with majority tonic phase (not TCS) according to examples of the present disclosure. The top figure is the X, Y, Z accelerometer trace and heart rate (BPM), as calculated through the PPG. The middle figure is model outputs for the tonic and clonic classifiers. The bottom figures is the accumulation filter. Note the overlap, and that there can be multiple clonic periods inside a TCS. Also please note that the classifier is lagged because of how the classifier is trained, the length of the window of data used for classification (20 seconds) and the fact that classifications are made causally. While there is both tonic and clonic activation, they do not happen in the correct manner to detect this segment as a seizure (accumulation will never reach the threshold).

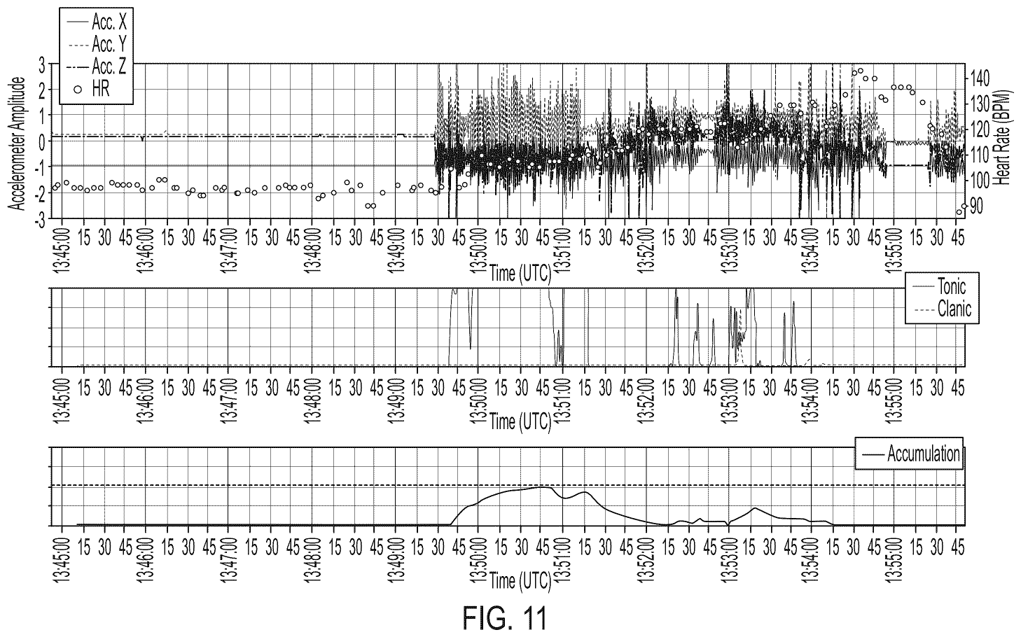

FIG. 11 shows data from a long single phase according to examples of the present disclosure. The top figure is the X, Y, Z accelerometer trace and heart rate (BPM), as calculated through the PPG. The middle figure is model outputs for the tonic and clonic classifiers. The bottom figure is the accumulation filter. Note in this case even though the phase is active for over a minute, the accumulation filter only asymptotically approaches 0.5. The threshold will never be met.

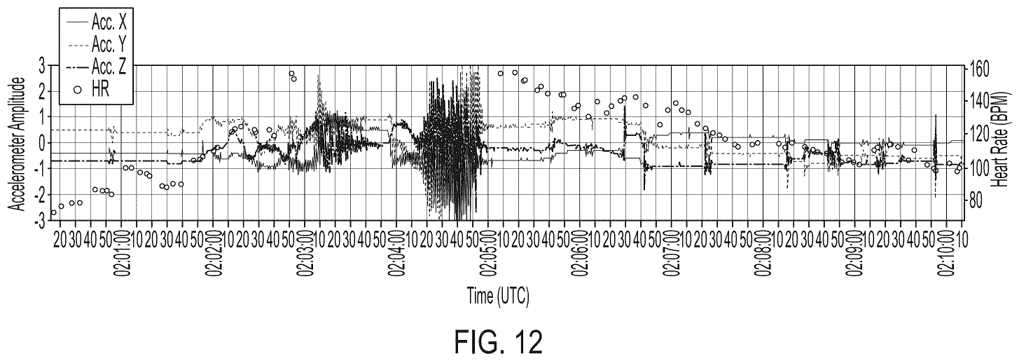

FIG. 12 shows data from a focal to bilateral tonic-clonic seizure (FBTCS) according to examples of the present disclosure. The figure shows the X, Y, Z accelerometer trace and heart rate (BPM), as calculated through the PPG. As shown in the example, the focal seizure lasts from 02:02:00 till 02:03:50, after which the actual TCS begins.

In some examples, in daily use, the method in the disclosed methods can use a refractory period of 10 minutes after a detection, within which no other detections may occur. This refractory period is present to ensure a single event does not cause multiple alerts. If a patient has a seizure that is detected, and caregivers come to provide aid, they will be present upon onset of the second seizure, should it occur within 10 minutes, so there is minimum safety compromise. The detector can be trained using a causal window looking a certain number of time points into the past. In many cases, a lag is present, especially for the end of the clonic phase. This is a result of how the model is trained, causality of the window, and length of window. Together, this will manifest as an offset on the detector output. This is clearly seen in FIG. 8 and FIG. 9. In some examples, FIG. 10 shows an example illustrating the necessity of both phases for detection of a tonic-clonic seizure. Note that only the tonic phase is active, and the seizure probably only approaches the chosen threshold, but never crosses it.

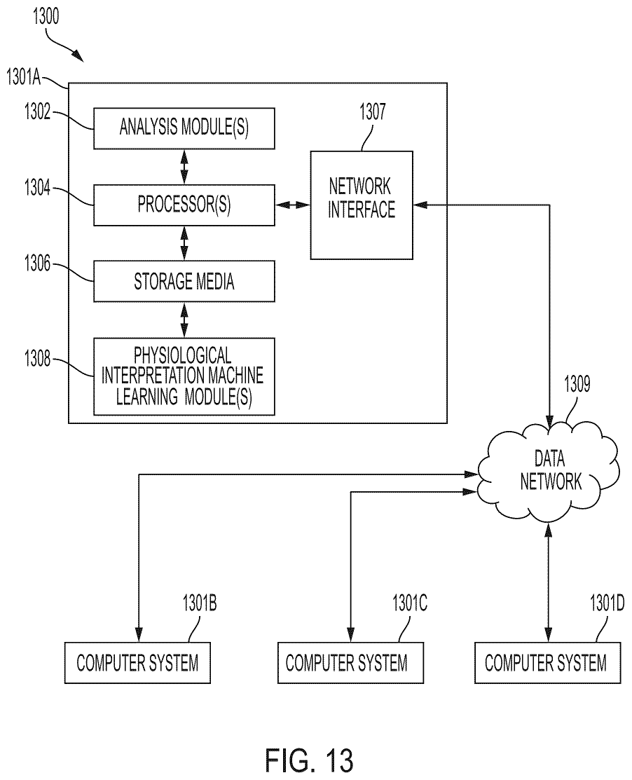

In some embodiments, any of the methods of the present disclosure may be executed by a computing system. FIG. 13 illustrates an example of such a computing system 1300, in accordance with some embodiments. The computing system 1300 may include a computer or computer system 1301A, which may be an individual computer system 1301A or an arrangement of distributed computer systems. The computer system 1301A includes one or more analysis module(s) 1302 configured to perform various tasks according to some embodiments, such as one or more methods disclosed herein. To perform these various tasks, the analysis module 1302 executes independently, or in coordination with, one or more processors 1304, which is (or are) connected to one or more storage media 1306. The processor(s) 1304 is (or are) also connected to a network interface 1307 to allow the computer system 1301A to communicate over a data network 1309 with one or more additional computer systems and/or computing systems, such as 1301B, 1301C, and/or 1301D (note that computer systems 1301B, 1301C and/or 1301D may or may not share the same architecture as computer system 1301A, and may be located in different physical locations, e.g., computer systems 1301A and 1301B may be located in a processing facility, while in communication with one or more computer systems such as 1301C and/or 1301D that are located in one or more data centers, and/or located in varying countries on different continents).

A processor can include a microprocessor, microcontroller, processor module or subsystem, programmable integrated circuit, programmable gate array, or another control or computing device.

The storage media 1306 can be implemented as one or more computer-readable or machine-readable storage media. The storage media 1306 can be connected to or coupled with a physiological interpretation machine learning module(s) 1308. Note that while in the example embodiment of FIG. 13 storage media 1306 is depicted as within computer system 1301A, in some embodiments, storage media 1306 may be distributed within and/or across multiple internal and/or external enclosures of computing system 1301A and/or additional computing systems. Storage media 1306 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions or located at a remote site from which machine-readable instructions can be downloaded over a network for execution.

It should be appreciated that computing system 1300 is only one example of a computing system, and that computing system 1300 may have more or fewer components than shown, may combine additional components not depicted in the example embodiment of FIG. 13, and/or computing system 1300 may have a different configuration or arrangement of the components depicted in FIG. 13. The various components shown in FIG. 13 may be implemented in hardware, software, or a combination of both hardware and software, including one or more signal processing and/or application specific integrated circuits.

Further, the steps in the processing methods described herein may be implemented by running one or more functional modules in an information processing apparatus such as general-purpose processors or application specific chips, such as ASICS, FPGAs, PLDs, or other appropriate devices. These modules, combinations of these modules, and/or their combination with general hardware are all included within the scope of protection of the invention.

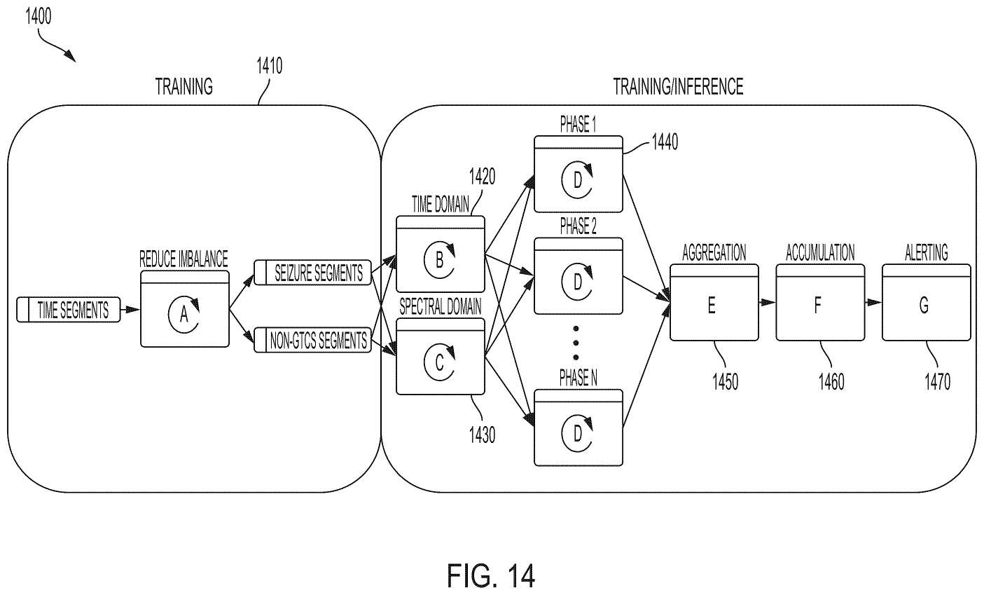

An exemplary embodiment of the method for physiological event detection and alerting is shown in FIG. 14, in which the method of training and inference 1400 is shown and the physiological event is an epileptic seizure. The method comprises sub-methods to automatically detect epileptic seizures and alert caregivers.

The imbalance reduction sub-method 1410 may identify seizure-like segments and reduces dataset imbalance. Datasets comprising information related to seizure events are prone to imbalances due to the rare and unexpected nature of seizure events, which leads to a low proportion of seizure events relative to the proportion of non-seizure events. In one embodiment, the imbalance reduction sub-method 1410 may comprise iteratively training unsupervised anomaly detection classifiers and performing inference on the dataset to identify non-seizure segments that are difficult to classify. In one embodiment, the anomaly detection method may comprise a One Class Support Vector Machine (OCSVM) technique. In another embodiment, the anomaly detection method may comprise a Support Vector Data Description (SVDD) technique. In another embodiment, the anomaly detection method may comprise an Extended Isolation Forest (IF) technique. In an exemplary embodiment, the anomaly detection method may comprise an Isolation Forest (IF) technique. In another embodiment, the anomaly detection method may comprise a combination of OCSVM, SVDD, Extended IF, and/or IF. The one or more anomaly detection classifier may significantly reduce dataset imbalance and allow for the use of supervised classifiers. The imbalance reduction sub-method 1410 may comprise a recurring sub-method. The imbalance reduction sub-method 1410 may output a balanced dataset.

The time domain sub-method 1420 may use the balanced dataset, an output of the imbalance reduction sub-method 1410, to either explicitly or implicitly generate time-domain features useful in identifying characteristics of input bio signals for characteristic phases of an epileptic seizure. This time domain sub-method 1420 can comprise a manual feature extraction method or a machine learning or deep learning method that will implicitly identify time domain features. While there are characteristic features that occur in epileptic seizures, they are not exclusive to epileptic seizures and may occur in non-seizure segments. In one embodiment, the time domain sub-method 1420 may comprise a deep learning feature extraction method for identification of characteristic implicit features of the tonic phase of an epileptic seizure from input bio signals such as movement (from data obtained from an accelerometer) and heart rate (from data obtained from a PPG). However, tonic phases may also occur in non-seizure segments. The time domain sub-method 1420 may comprise a recurring sub-method.

The spectral domain sub-method 1430 may use the balanced dataset, an output of the imbalance reduction sub-method 1410, to either explicitly or implicitly identify spectral-domain features useful in identifying characteristics of input bio signals for characteristic phases of an epileptic seizure. This spectral domain sub-method 1430 can comprise a manual feature extraction method or a machine learning or deep learning method that will implicitly identify spectral domain features. While there are characteristic features that occur in epileptic seizures, they are not exclusive to epileptic seizures and may occur in non-seizure segments. In one embodiment, the spectral domain sub-method 1430 may comprise a Fan Chirp Transform to identify the descending chirp from input bio signals. This descending chirp may be characteristic to the clonic phase of an epileptic seizure. However, descending chirps may also occur in non-seizure segments. The spectral domain sub-method 1430 may comprise a recurring sub-method.

The characteristic phase sub-method 1440 may comprise a characteristic phase classifier that is trained on the outputs of the time domain sub-method 1420 and spectral domain sub-method 1430 and may identify a characteristic phase of an epileptic seizure. There is no limit on the number of characteristic phases and hence characteristic phase classifiers that may exist in this characteristic phase sub-method 1440. In one embodiment, each characteristic phase classifier may output a confidence value corresponding to whether the characteristic phase is present in a segment of data. The output confidence value may, but does not necessarily, determine whether a given segment is a seizure. For example, a non-seizure segment may have a high confidence value in one or more characteristic phases. The characteristic phase sub-method 1440 may describe classifiers implemented as any classification method (i.e., supervised, unsupervised, or rules-based). In one embodiment, there may be two characteristic phases that are identified in an epileptic seizure, the tonic phase and the clonic phase. The classifiers for both of these phases may be implemented as deep learning models. For the tonic phase classifier, the characteristic phase sub-method 1440 may be trained end-to-end together with the time domain sub-method 1420 and the spectral domain sub-method 1430. For the clonic phase classifier, the characteristic phase sub-method 1440 may be trained independently from the outputs from the time domain sub-method 1420 and spectral domain sub-method 1430. The characteristic phase sub-method 1440 may comprise a recurring sub-method.

The aggregation sub-method 1450 may comprise an aggregation of the outputs from each characteristic phase classifier described in the characteristic phase sub-method 1440 in the form of an aggregate confidence score for each characteristic phase classifier. The multiple characteristic phases in the characteristic phase sub-method 1440 may ensure that the detector captures time segments that contain characteristics specific to epileptic seizures as opposed to other movements. In one embodiment, the aggregation sub-method 1450 may be implemented as a mean of the confidence value outputs from the characteristic phase sub-method 1440. In another embodiment, the aggregation sub-method 1450 may be implemented as a weighted sum of the confidence value outputs from the characteristic phase sub-method 1440. Weights can be calculated by performing a grid search over all possibilities (for example, in increments of 10%), and simulating inference of the time series data (including seizures and non-seizures). The weights that result in the best performance are selected. In one non-limiting example, the weights that resulted in the best performance are 50% for tonic phase and 50% for clonic phase. In another embodiment, the aggregation sub-method 1450 may be implemented as an arithmetic expression of the confidence value outputs from the characteristic phase sub-method 1440. In another embodiment, the aggregation sub-method 1450 may be implemented as a probabilistic graphical model such as Hidden Markov Models, Conditional Random Fields, or both. In another embodiment, the aggregation sub-method 1450 may be implemented as one or more of: a mean, a weighted sum, an arithmetic expression, or a probabilistic graphical model. For example, in the probabilistic graphical method, as well as in the deep learning method, the confidence outputs over time from the phase detectors can be used as inputs to the PGM or DL model. The model will identify the temporal characteristics associated with tonic clonic seizures and phase confidences over time to make a decision.

The accumulation sub-method 1460 may accumulate the aggregate confidence scores from the characteristic phase classifiers to ensure that any transient segments with high confidence do not prematurely trigger a detection. In one embodiment, this accumulation sub-method 1460 may comprise a first order infinite impulse response (IIR) filter. For example, the IIR filter is a low-pass filter that can be implemented in a real-time recursive manner by using y(n)=a*y(n−1)+b*x(n), where the values of a=0.95, b=0.05 are selected In another embodiment, the accumulation sub-method 1460 may comprise any accumulation method, including deep learning techniques such as a Recurrent Neural Network, a Long Short Term Memory Network, a Gated Recurrent Unit Network, a Temporal Convolutional Network, a Convolutional Neural Network, a Multi-Layer Perceptron, or combinations thereof. For example, the deep learning techniques can be configured to be similar to the scenario where the PGM for confidence outputs are used. The input to the DL (or PGM) model can be the confidence output of the previous stage (aggregation), and the output values, over a specified time window, can be input to the model. The model creates a temporal association between the aggregated time and a seizure detection.

The alerting sub-method 1470 may comprise an alerting system that is triggered once the output of the accumulation sub-method 1460 reaches a particular value, also known as a trigger value or threshold. In one embodiment, the alerting system may provide a potential physiological event alert on a user interface of a wearable device worn by the user. In another embodiment, the potential physiological alert may be provided to the user, a caregiver, a healthcare provider, a legal guardian, or combinations thereof.

Physiological interpretations, models, and/or other interpretation aids may be refined in an iterative fashion; this concept is applicable to embodiments of the present methods discussed herein. This can include use of feedback loops executed on an algorithmic basis, such as at a computing device (e.g., computing system 1300, FIG. 13), and/or through manual control by a user who may make determinations regarding whether a given step, action, template, model, or set of curves has become sufficiently accurate for the evaluation of the signal(s) under consideration.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. Moreover, the order in which the elements of the methods are illustrated and described may be re-arranged, and/or two or more elements may occur simultaneously. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A method for physiological event detection and alerting, the method comprising:
obtaining, from one or more biometric sensors, a set of biometric sensor data from a user;
generating, by one or more hardware processors, a set of processed biometric sensor data from the set of biometric sensor data;
generating, by the one or more hardware processors, a set of features from the processed biometric sensor data which are associated with one or more characteristic physiological event phase, wherein data based on an association between the set of features and the one or more characteristic physiological event phase is stored in one or more non-transitory storage media;
determining from the set of generated features, the set of processed biometric sensor data, or both the set of generated features and the processed biometric sensor data using the one or more hardware processors, a confidence score for each characteristic physiological event phase of the one or more characteristic physiological event phase indicating a presence of that phase in a data segment;
determining, from a relation between the confidence score of each of the characteristic physiological event phase using the one or more hardware processors, a final confidence score indicating an occurrence of a physiological event based on a relation between the one or more physiological event phase confidence scores;
determining, from an accumulation of final confidence scores using the one or more hardware processors, a cumulative confidence score indicating an occurrence of a particular physiological event, wherein the physiological event comprises one or more characteristic physiological event phases; and
providing, by the one or more hardware processors, a potential physiological event alert based on the cumulative confidence score.

2. The method of claim 1, wherein the one or more biometric sensors comprise an accelerometer and a photoplethysmography (PPG) sensor.

3. The method of claim 1, wherein the one or more biometric sensors are incorporated into a wearable device comprising of a wristwatch or glasses.

4. The method of claim 1, further comprising:
processing the set of biometric sensor data to produce the set of processed biometric sensor data;
reducing a data set imbalance between physiological events and non-physiological events in the processed biometric sensor data by iteratively training and using one or more models to identify anomalous segments in non-physiological event biometric sensor data to produce a balanced dataset, wherein the one or more models comprise one or more anomaly detection methods; and
using the balanced dataset to train one or more classifiers for each characteristic physiological event phase that produces the confidence score for each characteristic physiological event phase.

5. The method of claim 1, wherein the set of features from the processed biometric sensor data that are generated use techniques comprising one or more of a time domain feature extraction or a frequency domain feature extraction.

6. The method of claim 1, wherein the relation between the confidence scores determining the final confidence score comprises techniques of aggregating the confidence scores comprising a mean of the confidences scores, a weighted sum of the confidence scores, an arithmetic expression of the confidence scores, a probabilistic graphical model, or combinations thereof.

7. The method of claim 1, wherein the accumulation of final confidence scores to generate the cumulative confidence score comprises a first order infinite impulse response (IIR) filter.

8. The method of claim 1, wherein the potential physiological event alert is provided on a user interface of a wearable device worn by the user.

9. The method of claim 1, wherein the potential physiological event alert is provided to one or more of the user, a caregiver, a healthcare provider, or a legal guardian.

10. The method of claim 1, wherein a physiological event is any event that causes one or more characteristic patterns that can be identified through one or more of the biometric sensors, these events comprising: epileptic seizures, syncope, psychogenic non-epileptic seizures, movement disorders, or combinations thereof.

11. The method of claim 1, wherein the physiological event comprises a neurological event, a cardiac event, or combinations thereof.

12. The method of claim 11, wherein the neurological event is a seizure.

13. A system for physiological event detection and alerting, the system comprising:
one or more biometric sensors that capture, record, or both capture and record biosensor data from a user;
one or more hardware processors;
one or more non-transitory computer readable media that stores instructions, that when executed by the one or more hardware processors, perform a method of physiological detection and alerting comprising:
obtaining, from one or more biometric sensors, a set of biometric sensor data from a user;
generating, by one or more hardware processors, a set of processed biometric sensor data from the set of biometric sensor data;
generating, by the one or more hardware processors, a set of features from the processed biometric sensor data which are associated with one or more characteristic physiological event phase, wherein the association between the set of features and the one or more characteristic physiological event phase is stored in one or more non-transitory storage media;
determining from the set of generated features, the set of processed biometric sensor data, or both the set of generated features and the processed biometric sensor data using the one or more hardware processors, a confidence score for each characteristic physiological event phase of the one or more characteristic physiological event phase indicating a presence of that phase in a data segment;
determining, from a relation between the confidence score of each of the characteristic physiological event phase using the one or more hardware processors, a final confidence score indicating an occurrence of a physiological event based on a relation between the one or more physiological event phase confidence scores;
determining, from an accumulation of final confidence scores using the one or more hardware processors, a cumulative confidence score indicating an occurrence of a particular physiological event, wherein the physiological event comprises one or more characteristic physiological event phases;
providing, by the one or more hardware processors, a potential physiological event alert based on the cumulative confidence score; and
providing a user interface that provides the potential physiological event alert.

14. The system of claim 13, wherein the one or more biometric sensors comprise an accelerometer and a photoplethysmography (PPG) sensor.

15. The system of claim 13, wherein the one or more biometric sensors are incorporated into a wearable device comprising of a wristwatch or glasses.

16. The system of claim 13, wherein the method further comprising:
processing the set of biometric sensor data to produce the set of processed biometric sensor data;
reducing a data set imbalance between physiological events and non-physiological events in the processed biometric sensor data by iteratively training and using one or more models to identify anomalous segments in non-physiological event biometric sensor data to produce a balanced dataset, wherein the one or more models comprise one or more anomaly detection methods; and
using the balanced dataset to train one or more classifiers for each characteristic physiological event phase that produces the confidence score for each characteristic physiological event phase.

17. The system of claim 13, wherein the set of features from the processed biometric sensor data that are generated use techniques comprising one or more of a time domain feature extraction or a frequency domain feature extraction.

18. The system of claim 13, wherein the relation between the confidence scores determining the final confidence score comprises techniques of aggregating the confidence scores comprising a mean of the confidences scores, a weighted sum of the confidence scores, an arithmetic expression of the confidence scores, a probabilistic graphical model, or combinations thereof.

19. The system of claim 13, wherein the accumulation of final confidence scores to generate the cumulative confidence score comprises a first order infinite impulse response (IIR) filter.

20. The system of claim 13, wherein the potential physiological event alert is provided on a user interface of a wearable device worn by the user.

21. The system of claim 13, wherein the potential physiological event alert is provided to one or more of the user, a caregiver, a healthcare provider, or a legal guardian.

22. The system of claim 13, wherein a physiological event is any event that causes one or more characteristic patterns that can be identified through one or more of the biometric sensors, these events comprising: epileptic seizures, syncope, psychogenic non-epileptic seizures, movement disorders, or combinations thereof.

23. The system of claim 13, wherein the physiological event comprises a neurological event, a cardiac event, or combinations thereof.

24. The system of claim 23, wherein the physiological event is a seizure.
